(19) United States
(12) Patent Application Publication
(10) Pub. No.: US 2004/0228921 A1
(43) Pub. Date: Nov. 18, 2004

(54) Compositions and methods for delivery of therapeutic agents

(76) Inventors: Dipak K. Chowdhury, Lexington, KY (US); B. Ram Murty, Lexington, KY (US); Santos B. Murty, Lexington, KY (US)

Correspondence Address: George David McClure, Jr., P.P. Box 21902, Lexington, KY, 40522, US

(21) Appl. No.: 10/780,540

(22) Filed: Feb. 17, 2004

Related U.S. Application Data
(63) Continuation-in-part of application No. 10/694,377, filed on Oct. 27, 2003.
(60) Provisional application No. 60/447,413, filed on Feb. 14, 2003.
(60) Provisional application No. 60/447,414, filed on Feb. 14, 2003.

(51) Int. Cl. A61K 9/127; A61K 9/14

(52) U.S. Cl. 424/488

(57) ABSTRACT

Disclosed are pharmaceutical formulations for the ophthalmic delivery of tetrahydrocannabinol.

### CROSS-REFERENCE TO RELATED APPLICATIONS

This application is a continuation-in-part of application Ser. No. 10/694,377, filed Oct. 27, 2003.

### PRIORITY

Priority is claimed on the basis of provisional application Nos. 60/447,413 and 60/447,414, filed Feb. 14, 2003, which are fully incorporated herein by reference in their entirety.

### STATEMENT REGARDING FEDERAL SPONSORSHIP

Not applicable

### FIELD OF THE INVENTION

The invention relates to compositions and methods for delivery of therapeutic agents.

### BACKGROUND OF THE INVENTION

Surgical site infections-can be problematic, risky, and at times expensive. It has been estimated that surgical site infections lead to an annual increased expenditure of $3.3 billion (measured in 1992 dollars) [Quinn, Francis B., et al., “Microbiology, Infections and Antibiotic Therapy,” Grand Rounds Presentation, UTMB Dept. of Otolaryngology, Mar. 2000]. The need for lessening the probability of surgical site infection is reflected in reports concerning morbidity and mortality associated with arthroscopic knee surgery. At the time of preparation of the present application, links to web pages showing summaries of data concerning outpatient and inpatient surgeries can be found on the internet at www.cdc.gov/nchs/fastats.

Antibiotics have traditionally been delivered in dosage forms for oral or parenteral administration. These traditional delivery forms may provide excellent results when used for therapy or treatment of an active systemic infection, but these traditional delivery forms are in general not as effective when utilized to prevent localized infection at a surgical site. This is because the problem addressed by traditional delivery forms of antibiotics for the treatment of active systemic infection may be distinct from the problem addressed by delivery forms of antibiotics for the prevention of localized infection at a surgical site.

To be effective at preventing surgical site infection, oral or parenteral antibiotics must in general be administered prior to bacterial contamination of the surgical site, which in general requires administration before the surgical procedure. Where used afterwards, there is in general no beneficial effect when oral or parenteral antibiotics are administered more than three hours after surgery. Furthermore, where oral or parenteral antibiotics are administered prior to surgery in order to prevent surgical site infection, administration is required for at least five days after surgery, although standard practice typically requires a postoperative course of ten days.

Certain studies establish that there are significant benefits in the form of reduced infection rates associated with localized application of antibiotics [Polk, Hiram C., et al., “Prophylactic Antibiotics in Surgery and Surgical Wound Infections,” Dept. of Surgery, University of Louisville, 2000, citing Bergamini et al., “Combined Topical and Systemic Antibiotic Prophylaxis in Experimental Wound Infection,” Am J Surg., 1984; 147:753-756]. For instance, antibiotic powders, pastes, and aqueous solutions rinsed into incisions prior to closure have been found to be more effective than oral or parenteral antibiotics at preventing surgical site infection. Similarly, it has been found that surgical site infection rates were significantly reduced when patients' incisions were rinsed with an antibiotic solution for three days following surgery.

Studies therefore establish that localized application of an antibiotic at a surgical site can prevent localized infection at that site. There is a recognized need in the medical community for a preventive antibiotic formulation that may be applied locally at a surgical site. In addition, there is a recognized need among veterinarians and dentists for a preventive antibiotic formulation that may be applied locally at a surgical site. It would also be beneficial to deliver directly to the surgical site an analgesic or a local anesthetic for pain management, or, more generally, to deliver directly to an arbitrary site in a vertebrate subject a therapeutic agent needed for the prevention, treatment, lessening or amelioration of a condition which it is desired to prevent, treat, lessen or ameliorate in the subject.

### DESCRIPTION OF THE INVENTION

The invention provides pharmaceutical compositions useful for application or delivery of therapeutic agents, and methods of using the compositions.

For example, a composition according to the invention is useful for application of a therapeutic agent to, or contact of an agent with, an exposed surgical wound. For example, a composition according to the invention provides a dosage form possessing a bioadhesive property (texture).

In an embodiment, a composition according to the invention possesses substantially greater viscosity at about 37 degrees Celsius than at about 20 degrees Celsius.

In an embodiment, a composition according to the invention provides a sustained-release dosage form for a therapeutic agent, such as an antibiotic or an analgesic.

When used in connection with the invention, a “therapeutic agent” refers to a composition used for (a) the treatment of, therapy of, prophylaxis of, lessening the severity of, amelioration of, or forestalling (b) an injury, a disease, an infection, discomfort, pain, or a malady in a vertebrate. For example, a therapeutic agent comprises a composition known in the art to be a drug.

In an embodiment, the invention provides a medicinal substance comprising a composition possessing a viscosity that, at least within a portion of a certain range of temperatures, increases as the temperature of the composition increases. In a preferred embodiment, the certain range of temperatures is from about 15 degrees Celsius below the body temperature of a vertebrate in which it is desired to deliver a therapeutic agent to about the body temperature of the vertebrate.

In a preferred embodiment, the invention provides a composition useful for the topical administration of a drug to the skin of a vertebrate to which it is desired to administer the drug.

For example, an analgesic drug for topical administration according to the invention is methylsalicylate, menthol, camphor, methylnicotinate, triethanolamine salicylate, glycol salicylate, or salicylamine.

For example, an antifungal drug for topical administration according to the invention is tolnaftate, undecylenic acid, salicylic acid, zinc undecylenate miconazole, or thiabendazole.

For example, an antiviral drug for topical administration according to the invention is acyclovir or interferon.

For example, an anesthetic drug for topical administration according to the invention is procaine hydrochloride or lidocaine hydrochloride.

For example, an antimicrobial drug for topical administration according to the invention is iodine, povidone iodine, benzalkonium chloride or chlorhexidine gluconate.

For example, an antibacterial drug for topical administration according to the invention is a member of the group consisting of beta-lactam antibiotics, tetracyclines, chloramphenicol, neomycin, gramicidin, bacitracin, sulfonamides, aminoglycoside antibiotics, tobramycin, nitrofurazone, nalidixic acid and analogs, the antimicrobial combination of fludalanine/pentizdone, mafenide acetate, silver sulfadiazine, and nitrofurazone.

For example, an anti-inflammatory drug for topical administration according to the invention is a member of the group consisting of cortisone, hydrocortisone, betamethasone, dexamethasone, fluocortolone, prednisolone, triamcinalone, indomethacine, sulindac and its salts and corresponding sulfide.

For example, an antidermoinfective drug for topical administration according to the invention is a member of the group consisting of bifonazole, siccanin, bisdequalinium acetate, clotrimazole, salicylic acid, sulfamethoxazole sodium, erythromycin and gentamicin sulfate.

For example, an anti-inflammatory drug for topical administration according to the invention is a member of the group consisting of indomethacin, ketoprofen, betamethasone valerate and fluocinolone acetonide, diphenhydramine, cortisone, hydrocortisone, betamethasone, dexamethasone, fluocortolone, prednisolone, triamcinolone, indometnacin, sulinodac and its salts and corresponding sulfide.

For example, a miotic drug for topical administration according to the invention is a member of the group consisting of pilocarpine hydrochloride and carbachol.

For example, an antifungal drug for ophthalmic administration according to the invention is a member of the group consisting of amphotericin B, nystatin, flucytosine, natamycin and miconazole.

For example, an antiviral drug for ophthalmic administration according to the invention is a member of the group consisting of acyclovir, 5-iodo-2′-deoxyuridine (IDU), adenosine arabinoside (Ara-A), trifluorothymidine, interferon, and interferon-inducing agents such as poly I:C.

For example, an anesthetic drug for ophthalmic administration according to the invention is a member of the group consisting of lidocaine hydrochloride, oxybuprocaine hydrochloride, procaine, benzocaine, xylocaine, etidocaine, cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, and mepivacaine.

For example, an antibiotic drug for ophthalmic administration according to the invention is a member of the group consisting of amphotericin B, norfloxacin, miconazole nitrate, ofloxacin, idoxuridine, chloramphenicol, colistin sodium methanesulfonate, carbenicillin sodium, beta-lactam antibiotics, cefoxitin, n-formanidolthienamycin and other thienamycin derivatives, tetracyclines, neomycin, carbenicillin, colistin, penicillin G, polymyxin B, vancomycin, cefazolin, cephaloridine, chibrorifamycin, gramicidin, bacitracin and sulfonamides.

For example, an aminoglycoside drug for ophthalmic administration according to the invention is a member of the group consisting of gentamycin, kanamycin, amikacin, sisomicin, tobramycin, nalidixic acid and its analogs such as norfloxacin, fluoroalanine/pentizidone, nitrofurazones and analogs thereof.

For example, an antibiotic/antiinflammatory combination drug for ophthalmic administration according to the invention is a member of the group consisting of neomycin sulfate and dexamethasone sodium phosphate, timolol maleate and aceclidine, nalidixic acid and its analogs such as norfloxacin and the antimicrobial combination fluoroalanine/pentizidone, nitrofurazones and analogs thereof.

For example, an antiallergic drug for ophthalmic administration according to the invention is a member of the group consisting of 3′-(1H-tetrazol-5-yl)oxanilic acid(MTCC), ketotifen fumarate and sodium cromoglicate.

For example, an antiinflammatory drug for ophthalmic administration according to the invention is a member of the group consisting of cortisone, hydrocortisone, hydrocortisone acetate, betamethasone, dexamethasone, dexamethasone sodium phosphate, prednisone, methylprednisolone, medrysone, fluorometholone, prednisolone, prednisolone sodium phosphate, triamcinolone, indomethacin, sulindac, its salts and its corresponding sulfides, and analogs thereof, glycyrrhizinate dipotassium, lysozyme chloride, diclofenac sodium, pranoprofen, cortisone acetate, azulene, allantoin and .epsilon.-aminocaproic acid.

For example, an anticholinergic or miotic drug for ophthalmic administration according to the invention is a member of the group consisting of echothiophate, pilocarpine, physostigmine salicylate, diisopropylfluorophosphate, epinephrine, dipivalopylepinephrine, neostigmine, echothiopate iodide, demecarium bromide, carbamoyl choline chloride, methacholine, bethanechol, and analogs thereof.

For example, an antiglaucoma or anticataract drug for ophthalmic administration according to the invention is a member of the group consisting of timolol maleate, carteolol hydrochloride, glutathione, pirenoxine, R-timolol, and a combination of timolol or R-timolol with pilocarpine.

For example, a mydriatic drug for ophthalmic administration according to the invention is a member of the group consisting of atrophine, homatropine, scopolamine, hydroxyamphetamine, ephedrine, cocaine, tropicamide, phenylephrine, cyclopentolate, oxyphenonium, eucatropine, and analogs thereof.

For example, an antihistamine drug for ophthalmic administration according to the invention is a member of the group consisting of chlorpheniramine maleate and diphenhydramine hydrochloride.

For example, an antiparisitic or antiprotozoal drug for ophthalmic administration according to the invention is a member of the group consisting of ivermectin, pyrimethamine, trisulfapidimidine, clindamycin and corticosteroids.

For example, a surgical adjunct therapeutic agent for ophthalmic administration according to the invention is a member of the group consisting of proteases such as alpha-chymotrypsin and dispase and polysaccharide hydrolases such as hyaluronidase.

The invention provides a composition for administration of a therapeutic agent into or delivery of a therapeutic agent to a body cavity of a mammal, such as rectum; urethra, nasal cavity, vagina, auditory meatus, oral cavity or buccal pouch. Any one or more of a wide variety of therapeutic agents are administered or delivered through use of a composition according to the invention. Examples of therapeutic agents for administration or delivery through use of a composition according to the invention are enumerated below:

Analgesics such as methyl salicylate, menthol, camphor, methyl nicotinate, triethanolamine salicylate, glycol salicylate, salicylamide, morphine sulfate, codeine sulfate, meperidine, and nalorphine, aspirin, indomethacin, sulindac, phenylbutazone, ibuprofen, and acetaminophen;

Antivirals such as acyclovir and interferon;

Anesthetics such as lidocaine, benzocaine, dibucaine, procaine, and xylocaine;

Antifungals such as miconazole nitrate, candicidin, nystatin, clotrimazole, and metronidazole;

Dermatics for purulence such as sulfisoxazole, kanamycin, tobramycin and erythromycin;

Antimicrobials such as beta-lactams, such as cefoxitin, n-formamidoyl thienamycin and other thienamycin derivatives, tetracyclines, chloramphenicol, neomycin, gramicidin, bacitracin, sulfonamides; aminoglycoside antibiotics such as gentamycin, kanamycin, amikacin, sisomicin and tobramycin; nalidixic acids and analogs such as norfloxacin and the antimicrobial combination of fludalanine/pentizidone, nitrofurazones, carbenicillin, colistin, penicillin G, polymyxin B, vancomycin, cefazolin, cephaloridine, chibrorifamycin, penicillin, tetracycline, chloramphenicol, minocycline, doxycycline, vanomycin, bacitracin, kanamycin, neomycin, gentamycin, erythromicin and cephalosporins;

Antibiotic/antiinflammatory combinations such as neomycin sulfate-dexamethasone sodium phosphate;

Anti-glaucoma concomitant therapeutic agents such as timolol maleate-aceclidine;

Anti-pyretics such as aspirin (salicylic acid), indomethacin, sodium indomethacin trihydrate, salicylamide, naproxen, colchicine, fenoprofen, sulindac, diflunisal, diclofenac, indoprofen and sodium salicylamide;

Anti-inflammatories such as cortisone, hydrocortisone, hydrocortisone acetate, betamethasone, dexamethasone, dexamethasone sodium phosphate, prednisone, methylprednisolone, medrysone, fluorometholone, fluocortolone, prednisolone, prednisolone sodium phosphate, triamcinolone, indomethacin, sulindac, its salts and its corresponding sulfide;

Miotics such as echothiophate, pilocarpine, physostigmine salicylate, diisopropylfluorophosphate, epinephrine, neostigmine, carbachol, methacholine, bethanechol, and dipivolyl epinephraine;

Antihistamines such as pyrilamine, chlorpheniramine, tetrahydrazoline, and diphenhydramine hydrochloride;

Adrenal hormone preparations such as dexamethasone, triamcinolone and hydrocortisone;

Hypnotics and sedatives such as diazepam;

Antiparasitic compounds and/or anti-protozoal compounds such as ivermectin, pyrimethamine, trisulfapyrimidine, clindamycin and corticosteroid preparations;

Adrenergic agonists and/or antagonsists such as epinephrine and an epinephrine complex, or prodrugs such as bitartrate, borate, hydrochloride and dipivefrine derivatives;

Carbonic anhydrase inhibitors such as acetazolamide, dichlorphenamide, 2-(p-hydroxyphenyl)-thio thiophenesulfonamide, 6-hydroxy-2-benzothiazolesulfonamide, and 6-pivaloyloxy-2-benzothiazolesulfonamide;

Muscle relaxants such as succinylcholine chloride, danbrolene, cyclobenzaprine, methocarbomol, and diazepam;

Chelating agents such as ethylenediamine tetraacetate (EDTA) and deferoxamine;

Peptides and proteins such as atrial natriuretic factor, calcitonin-gene related factor, lutinizing hormone, releasing hormone, neuroterisin, vasoactive intestinal peptide, vasopressin, cyclosporine, interferon, substance P enkephalins, epidermal growth factor, eye derived growth factor, fibronectin, insulin-like growth factor, and mesodermal growth factor;

Immunosuppressive agents, antineoplastics and anti-metabolites such as adriamycin, asparaginase, methotrexate, cyclophosphamide, 6-mercaptopurine, azathioprine, cisplatin, prednisone, hydroxyprogesterone, medroxyprogesterone acetate, megestrol acetate, diethylstilbestrol, testosterone propionate, fluoxymesterone, vinblastine, vincristine, vindesine, daunorubicin, doxorubicin, hydroxyurea, procarbazine, aminoglutethimide, mechlorethamine, cyclophosphamide, melphalan, uracil mustard, chlorambucil, busulfan, carmustine, lomustine, dacarbazine (DTIC: dimethyltriazenomidazolecarboxamide), methotrexate, 5-fluorouracil, cytarabine, cytosine arabinoside, and thioguanine.

The invention thus provides a composition comprising, by mass, from about 1% to about 3% therapeutic agent, from about 0.05% to about 0.5% carbopol, from about 0.1% to about 0.5% hydroxypropylmethylcellulose, from about 14% to about 20% Lutrol F127, from about 13% to about 20% Lutrol F68, from about 0.1% to about 0.5% trolamine 10% w/v aqueous solution, and water.

The invention provides a composition, by mass, 1% clindamycin, 20% poloxamer, and water.

The invention provides a composition comprising, by mass, 1% clindamycin, 0.5% HPMC, 15% poloxamer, and water,

The invention provides a composition comprising, by mass, 3% clindamycin, 0.3% carbopol, 15% poloxamer, 0.3% trolamine 10% w/v aqueous solution, and water.

A composition according to the invention is useful for treatment of, therapy of, prophylaxis of, lessening the severity of, amelioration of, or forestalling an injury, a disease, an infection, discomfort, pain, or a malady in a vertebrate.

The invention accordingly provides a method of forestalling infection in a vertebrate, comprising the step of administering to the vertebrate, at a site in or on the vertebrate where it is desired to forestall infection, a therapeutically effective amount of a composition according to the invention.

A composition according to the invention was prepared according to the following formula and found to be useful in the delivery or administration of a therapeutic agent, in this case, clindamycin:

| | |
|---|---|
| Lutrol F127 | 20% |
| Clindamycin | 1% |
| 0.1 M phosphate buffer | qs 100 g |

A further composition according to the invention was prepared according to the following formula and found to be useful in the delivery or administration of a therapeutic agent, in this case, clindamycin:

| | |
|---|---|
| Lutrol F127 | 20% |
| Clindamycin | 1% |

0.9% Sodium Chloride in Water qs 100 g

Yet a further composition according to the invention was prepared according to the following formula and found to be useful in the delivery or administration of a therapeutic agent, in this case, clindamycin:

| | |
|---|---|
| Lutrol F127 | 15% |
| Carbopol 934F | 0.1-0.5% |
| Clindamycin | 1% |
| Deionized water | qs 100 g |

Preliminary compositions were prepared and tested for the establishment of the properties of said compositions:

| | |
|---|---|
| Lutrol F127 | 15% |
| Lutrol F68 | 18% |
| Deionized water | qs 100 g |
| *** | |
| Lutrol F127 | 15% |
| Lutrol F68 | 18% |
| 0.9% NaCl in Water | qs 100 g |
| *** | |
| Lutrol F127 | 15% |
| Lutrol F68 | 18% |

0.1 M Phosphate Buffer pH 7.4 qs 100 g

Further preliminary compositions were prepared and tested for the establishment of the properties of said compositions:

| | |
|---|---|
| Lutrol F127 | 15% |
| HPMC | 0.1-0.5% |
| Deionized Water | qs 100 g |
| *** | |
| Lutrol F127 | 15% |
| HPMC | 0.3% |
| Carbomer | 0.3% |

0.1 M Phosphate Buffer pH 7.0 qs 100 g * * *

| | |
|---|---|
| Lutrol F127 | 15% |
| HPMC | 0.3% |
| Carbomer | 0.3% |
| 0.9% NaCl in Water | qs 100 g |
| *** | |
| Lutrol F127 | 15% |
| HPMC | 0.3% |
| Carbomer | 0.3% |
| Deionized Water | qs 100 g |

Further embodiments of the invention are as follows.

Embodiment: Narcotic Analgesics for Sublingual/Buccal and Transdermal Delivery: e.g., Fentanyl.

Solubility: 1000 mg/40 mL=25 mg/mL

Doses: 1.2-1.8 mg

Formulation:

| | |
|---|---|
| Lutrol F127 | 15% |
| Carbopol 934F | 0.1-0.5% |
| Fentanyl | 1% |
| Deionized water | qs 100 g |

Therefore, apply 0.12-0.18 g of formulation to obtain desired dose.

Embodiment: Steroidal Anti-Inflammatory for Topical and Ophthalmic Administration: e.g. Dexamethasone Sodium.

Solubility: 1 g/2 mL Freely-Soluble

Doses: 0.05-0.1% Applied Topically

Formulation:

| | |
|---|---|
| Lutrol F127 | 15% |
| Carbopol 934F | 0.1-0.5% |
| Dexamethasone sodium | 0.05-0.1% |
| Deionized water | qs 100 g |

Therefore, apply formulation to obtain desired dose in the eye.

Embodiment: Anti-Viral Agent for Topical: e.g. Acyclovir Sodium.

Solubility: 1 g/10 mL Water

Dose: 3.0% Topical

Formulation:

| | |
|---|---|
| Lutrol F127 | 15% |
| Carbopol 934F | 0.1-0.5% |
| Acyclovir Sodium | 3.0% |
| Deionized water | qs 100 g |

Embodiment: formulation for delivery of anesthetic: e.g., lidocaine.

Solubility—1 g/1 ml of water

Dose - - - 250 mg-350 mg/15ml

Formulation:

| | |
|---|---|
| Lutrol F127 | 15% |
| Hydroxypropylmethylcellulose | 0.1-0.5% |
| Lidocaine | 3.0% |
| Deionized water | qs 100 g |

Embodiment: formulation for delivery of narcotic analgesics: e.g., morphine sulphate. Formulation:

| | |
|---|---|
| Lutrol F127 | 15% |
| Hydroxypropylmethylcellulose | 0.1-0.5% |
| Morphine sulphate | 3.0% |
| Deionized water | qs 100 g |

Embodiment: formulation for delivery of ophthalmic antibiotic: e.g., ciprofloxacin hydrochloride

Dose - - - 100-200 mg twice daily

Formulation:

| | |
|---|---|
| Lutrol F127 | 15% |
| Hydroxypropylmethylcellulose | 0.1-0.5% |
| Ciprofloxacin lactate | 1.0% |
| Phosphate buffer pH 4.4 | qs 100 g |

Embodiment: formulation for delivery of mydriatic: e.g., atropine sulphate.

Solubility—1 gm/0.5 ml of water

Dose - - - 0.1-0.2 gm

Formulation:

| | |
|---|---|
| Lutrol F127 | 15% |
| Hydroxypropylmethylcellulose | 0.1-0.5% |
| Atropine sulphate | 1.0% |
| Phosphate buffer pH 4.4 | qs 100 g |

The following embodiments show the usefulness of the invention for the delivery of tetrahydrocannabinol, especially via the ocular or ophthalmic route.

As used in the following descriptions of embodiments, the term “Poloxamer” refers to any of the group consisting of polyoxyethylene-polyoxypropylene block copolymers known in the art; the term “Carbopol” refers to any of the group consisting of cross-linked poly (acrylic acid) polymers known in the art, said group comprising homopolymers and copolymers; and the term “cellulose ether” refers to any of the group consisting of cellulose ethers known in the art, said group including methylcellulose and hydroxypropyl methlylcellulose.

In a first embodiment, the invention provides a pharmaceutical composition comprising THC and a Poloxamer.

In a second embodiment, the invention provides a pharmaceutical composition according to the first embodiment, wherein the Poloxamer comprises Poloxamer 407.

In a third embodiment, the invention provides a pharmaceutical composition according to the first embodiment and further comprising a Carbopol.

In a fourth embodiment, the invention provides a pharmaceutical composition according to the first embodiment and further comprising a cellulose ether.

In a fifth embodiment, the invention provides a pharmaceutical composition according to the first embodiment and further comprising a Carbopol and a cellulose ether.

In a sixth embodiment, the invention provides a pharmaceutical composition according to the first embodiment, wherein the Poloxamer comprises any of the group consisting of Poloxamer 188, Poloxamer 237, and Poloxamer 407.

In a seventh embodiment, the invention provides a pharmaceutical composition according to the sixth embodiment, wherein the proportion of the Poloxamer in the composition is, by mass, from about 10% to about 20%.

In an eighth embodiment, the invention provides a pharmaceutical composition according to the second embodiment and further comprising any of the group consisting of Poloxamer 188 and Poloxamer 237.

In a ninth embodiment, the invention provides a pharmaceutical composition according to the third embodiment or the fifth embodiment, wherein (a) the Carbopol comprises any of the group consisting of Carbopol grade 910, Carbopol grade 934, Carbopol grade 940, Carbopol grade 941, Carbopol grade 974, Carbopol grade 981, Ultrez and Polycarbophil and (b) the proportion of the Carbopol in the composition is, by mass, from about 0.05% to about 0.5%.

In a tenth embodiment, the invention provides a pharmaceutical composition according to the fourth embodiment or the fifth embodiment, wherein (a) the cellulose ether possesses a viscosity from about 3 to about 100,000 millipascal-seconds (mPa×s) and (b) the proportion of the cellulose ether in the composition is, by mass, from about 0.05% to about 0.5%.

In an eleventh embodiment, the invention provides a pharmaceutical composition according to the first embodiment and further comprising any of the group consisting of Cremophore, N-methyl-2-pyrrolidone (Pharmasolve), Poloxamer 237, Propylene glycol and Polysorbate 80 (Tween 80).

In a twelfth embodiment, the invention provides a pharmaceutical composition according to the fifth embodiment, wherein (a) the Carbopol comprises any of the group consisting of Carbopol grade 910,. Carbopol grade 934, Carbopol grade 940, Carbopol grade 941, Carbopol grade 974, Carbopol grade 981, Ultrez and Polycarbophil; (b) the proportion of the Carbopol in the composition is, by mass, from about 0.05% to about 0.5%; (c) the cellulose ether possesses a viscosity from about 3 to about 100,000 mPa×s; (d) the proportion of the cellulose ether in the composition is, by mass, from about 0.05% to about 0.5%; and (e) the Poloxamer comprises any of the group consisting of Poloxamer 188, Poloxamer 237, and Poloxamer 407.

In a thirteenth embodiment, the invention provides a pharmaceutical composition according to the tenth embodiment, wherein the cellulose ether comprises any of the group consisting of methylcellulose and hydroxypropyl methylcellulose.

In a fourteenth embodiment, the invention provides a method of treating, preventing, ameliorating, lessening or mitigating glaucoma or increased intraocular pressure, the method comprising administering to a subject in need of said treating, preventing, ameliorating, lessening or mitigating a therapeutically effective amount of a pharmaceutical composition according to any of the foregoing embodiments.

Each of the foregoing numbered embodiments provides a formulation for delivery of tetrahydrocannabinol to the eye, for which there has been a long-felt need, as tetrahydrocannabinol is known in the art to be useful for the treatment or amelioration of glaucoma.

Each of the foregoing embodiments is merely exemplary and is not intended to limit the scope of the invention, which encompasses all equivalents of what is described herein and set forth in the following claims.

What is claimed is:

1. A pharmaceutical composition for ophthalmic delivery of THC, the composition comprising THC and a Carbopol, and wherein (a) the Carbopol comprises any of the group consisting of Carbopol grade 910, Carbopol grade 934, Carbopol grade 940, Carbopol grade 941, Carbopol grade 974, Carbopol grade 981, Ultrez and Polycarbophil and (b) the proportion of the Carbopol in the composition is, by mass, from about 0.05% to about 0.5%.

2. A pharmaceutical composition for ophthalmic delivery of THC, the composition comprising THC and a cellulose ether, and wherein (a) the cellulose ether possesses a viscosity from about 3 to about 100,000 millipascal-seconds (mPa×s) and (b) the proportion of the cellulose ether in the composition is, by mass, from about 0.05% to about 0.5%.

3. A pharmaceutical composition for ophthalmic delivery of THC, the composition comprising a Poloxamer, a Carbopol, and a cellulose ether, and wherein (a) the Carbopol comprises any of the group consisting of Carbopol grade 910, Carbop l grade 934, Carbopol grade 940, Carbopol grade 941, Carbopol grade 974, Carbopol grade 981, Ultrez and Polycarbophil; (b) the proportion of the Carbopol in the composition is, by mass, from about 0.05% to about 0.5%; (c) the cellulose ether possesses a viscosity from about 3 to about 100,000 mPa×s; (d) the proportion of the cellulose ether in the composition is, by mass, from about 0.05% to about 0.5%; and (e) the Poloxamer comprises any of the group consisting of Poloxamer 188, Poloxamer 237, and Poloxamer 407.

4. A composition according to claim 2, wherein the cellulose ether comprises any of the group consisting of methylcellulose and hydroxypropyl methylcellulose.

5. A method of treating, preventing, ameliorating, lessening or mitigating glaucoma or increased intraocular pressure, the method comprising administering to a subject in need of said treating, preventing, ameliorating, lessening or mitigating a therapeutically effective amount of a pharmaceutical composition according to claim 1.

6. A method of treating, preventing, ameliorating, lessening or mitigating glaucoma or increased intraocular pressure, the method comprising administering to a subject in need of said treating, preventing, ameliorating, lessening or mitigating a therapeutically effective amount of a pharmaceutical composition according to claim 2.

7. A method of treating, preventing, ameliorating, lessening or mitigating glaucoma or increased intraocular pressure, the method comprising administering to a subject in need of said treating, preventing, ameliorating, lessening or mitigating a therapeutically effective amount of a pharmaceutical composition according to claim 3.

8. A method of treating, preventing, ameliorating, lessening or mitigating glaucoma or increased intraocular pressure, the method comprising administering to a subject in need of said treating, preventing, ameliorating, lessening or mitigating a therapeutically effective amount of a pharmaceutical composition according to claim 4.
